(19) United States
(12) Patent Application Publication
(10) Pub. No.: US 2006/0047215 A1
(43) Pub. Date: Mar. 2, 2006

(54) Combined sensor assembly

(75) Inventors: Richard W. Newman, Auburn, NY (US); Allan I. Krauter, Skaneateles, NY (US); James P. Welch, Tigard, OR (US)

Correspondence Address: WALL MARJAMA & BILINSKI, 101 SOUTH SALINA STREET, SUITE 400, SYRACUSE, NY, 13202, US

(73) Assignee: Welch Allyn, Inc., Skaneateles Falls, NY (US)

(21) Appl. No.: 10/931,390

(22) Filed: Sep. 1, 2004

(51) Int. Cl. A61B 5/04; A61B 5/02

(52) U.S. Cl. 600/513; 128/903; 600/528

(57) ABSTRACT

A combined sensor assembly used in conjunction with a patient includes at least one electrical sensor that is capable of detecting electrical signals that are indicative of a physiological parameter. The at least one electrical sensor is coupled to the patient by means of an electrically conductive gel material. The sensor assembly further includes at least one acoustic sensor that is coupled to the patient using an acoustically conductive gel material. The conductive gel material used in conjunction with the at least one acoustic sensor and the at least one electrical sensor can be the same or a different material, wherein a transducer of the acoustic sensor and the acoustically conductive gel define an interface region that is essentially devoid of air.

### FIELD OF THE INVENTION

This invention relates to the field of patient vital signs monitoring, and in particular to a combined sensor assembly that integrates at least one electrical sensor capable of measuring electrical signals representative of a physiological parameter of a patient with at least one acoustic sensor, such as a microphone.

### BACKGROUND OF THE INVENTION

A number of known sensor assemblies have been made available in the field of remote monitoring, particularly the field of vital signs monitoring, in order to measure certain physiological parameters of a patient, such as, for example, electrical signals from a patient in the form of ECG (electrocardiogram) signals. To that end, a conventional sensor assembly 10 that is used for this purpose, such as depicted in FIG. 1(b), includes a plurality of electrodes 20 that are individually attached onto the chest 24 of a patient 23 in a pre-arranged configuration. Each of the electrodes 20, as shown in FIGS. 1(b) and 1(c), includes a transducer that gathers ECG electrical signals from the heart of the patient 23 and then relays the gathered signals via a series of connected cables 25 to a tethered ECG monitor 28 or chart recorder (not shown) for display. The electrodes 20 of the above assembly 10 are directly applied and electrically coupled to the skin of the patient 23 using an electrically conductive gel material that is disposed on the bottom facing side of each attached electrode. The electrodes are mechanically attached to the skin 51, FIG. 2, of the patient by an adhesive tape. Separate from the above assembly 10, heart-related and respiratory (e.g., lung) sounds can be detected using a dedicated stethoscope 30, as shown in FIG. 1(a), preferably a stethoscope that includes an acoustic transducer/microphone 34.

Applicants are presently aware of U.S. Patent Applications U.S. 2003/0176800A1 and U.S. 2003/0176801A1, each of which describe a combination assemblage that includes both an ECG electrode, as well as an acoustic microphone, that are arranged coaxially relative to one another. As is shown in FIG. 1 of the '800 publication, the microphone is disposed within the assemblage at the apex of a conically or bell-shaped collection volume that is formed above the ECG electrode portion thereof. The purpose of the collection volume according to the teachings of the patent is to focus and isolate the reception of audio sounds, such as respiration or heart-related sounds, by the acoustic transducer of the microphone, as is typically done for microphones of this type. The above reference further observes that the use of an electrically conductive gel used with the ECG electrode portion of the assembly assists in sealing the collection volume and further assists to prevent against inside/outside air flow relative to the collection volume.

### SUMMARY OF THE INVENTION

It is therefore a primary object of the present invention to improve the overall efficiency and design of vital signs monitoring systems.

It is another primary object of the present invention to provide an improved sensor assembly in order to provide improved ease in patient examination, increased efficiency and/or increased accuracy.

It is another primary object of the present invention to provide a low cost, reliable sensor that is suitable for attachment, for example, to the body of a patient.

It is another primary object of the present invention to provide improved acoustic performance for a sensor assembly, the assembly being insensitive to acoustic noise and preferably having a low-profile configuration.

Therefore and according to a preferred aspect of the present invention, there is provided a combined sensor assembly comprising:
at least one electrical sensor, said at least one electrical sensor being capable of measuring electrical signals representative of a physiological parameter of a patient and coupled by means of an electrically conductive gel material; and
at least one acoustic sensor, each said at least one acoustic sensor being coupled to said patient by means of an acoustically conductive gel material.

According to one embodiment of the present invention, the at least one acoustic sensor and the at least one electrical sensor are each coupled to the patient using the same conductive gel material, wherein the conductive gel material provides transmission characteristics so as to provide an effective acoustic impedance match to the skin in addition to providing electrical conductivity for the electrical sensor. Preferably, the at least one acoustic sensor comprises a microphone having an acoustic transducer that is directly coupled with the conductive gel material substantially without an intermediate air buffer, such as that described and required in the field, for example, in the preceding '800 publication.

The combined sensor assembly can be designed with the two sensors (electrical, acoustic) arranged either coaxially or laterally with respect to one another.

The herein described combined sensor assembly can include literally any form of physiological sensor that detects electrical activity of a patient (e.g., ECG, EEG, EMG, etc.) but can further include additional physiologic sensors in addition to the at least one electrical sensor, such as those capable of measuring, for example, body temperature, blood pressure, heart rate, blood glucose, blood oxygen saturation, and the like, these additional sensors not necessarily relying upon an electrical signal generated from the patient. Preferably, the combined sensor assembly can be configured for use in either a hard-wired or tethered version in order to transmit the generated signals from the contained sensors to a bedside monitor or to a hospital network. Alternatively, a miniature radio transceiver antenna, and embedded microprocessor can be added to the overall sensor assembly in order to permit wireless transmission of ECG and other physiological parametric data to a remote location. As such, the herein described sensor assembly can be used to monitor numerous patient vital signs, physical diagnoses, and/or molecular diagnoses, in which representative detected signals can be transmitted from the combined sensor assembly by either a wired or a wireless connection to a remote monitoring station or other site.

One advantage provided is that the combined sensor assembly of the present invention is fairly simple in design and is easily manufactured. The sensor assembly can be used in a conventional manner as to attachment to a patient, therefore no new training is required.

Another advantage provided by the present combined sensor assembly is that use of a conductive gel material with an integrated microphone or other form of acoustic sensor permits respiratory and heart-related sounds to be picked up more readily than known assemblies for this purpose and without requiring multiple and separate assemblies with good immunity to extraneous acoustic noise, such as that produced by chest hair. Another advantage is that a combined sensor assembly as described can be made cheaper than those previously known. A further advantage is that only a single gel can be required to effectively couple the assembly to the patient, the assembly thereby being easy to apply and use.

These and other objects, features and advantages will become readily apparent from the following Detailed Description that should be read in conjunction with the accompanying drawings.

### BRIEF DESCRIPTION OF THE DRAWINGS

FIG. 1(a) depicts a prior art stethoscope used in detecting respiratory and heart related sounds from a patient;

FIG. 1(b) depicts a prior art ECG monitoring assembly;

FIG. 1(c) depicts a bottom facing view of the electrode of the prior art monitoring assembly of FIG. 1(b);

FIG. 2 depicts a prior art combination ECG/stethoscope sensor assembly;

FIG. 3 is a side elevation view, shown in section, of a combined sensor assembly made in accordance with a first embodiment of the present invention;

FIG. 4 is a bottom view of a combined sensor assembly made in accordance with a second embodiment of the present invention;

FIG. 5 is a partial section view of the combined sensor assembly of FIG. 4 as taken through lines 5-5;

FIG. 6 is a perspective view of the combined sensor assembly of FIG. 4 in use with a patient;

FIGS. 7 and 7(a) represent alternative side elevational views of a combined sensor assembly made in accordance with a third embodiment of the present invention;

FIGS. 8(a) and 8(b) are partial perspective views of an acoustic sensor used for purposes of testing; and

FIGS. 9-14 are representative plots illustrating the relative performance of the acoustic sensor assembly of FIG. 8, based on various applied loads and use of acoustically conductive gel.

### DETAILED DESCRIPTION

The following description relates to a combined sensor assembly for use in monitoring a patient, the assembly comprising at least one electrical sensor capable of measuring an electrical signal representative of a physiological parameter of a patient and at least one integrated acoustic sensor that is made in accordance with certain preferred embodiments of the present invention. Throughout the discussion that follows, certain terms such as “top”, “bottom”, “lateral”, and the like are used to relate a frame of reference with regard to the accompanying drawings. These terms, however, should not viewed as overly limiting of the present invention, except where specifically indicated. In addition, the electrical sensor portion of the combined sensor assembly described herein is an ECG sensor assembly for detecting electrical signals from the heart of a patient. It will be readily apparent, however, that the herein described combined sensor assembly can be used in connection with literally any physiological parameter sensor that is capable of detecting an electrical signal relating to a patient, such as for example, EEG, EMG, and the like. From the following discussion it will also be readily apparent to those of sufficient skill in the field that additional physiological parameter sensors, whether electrical, acoustic, or other, can also be integrated into the present sensor assembly in combination with those discussed above for measurement of other patient vital signs such as body temperature, blood glucose, respiration rate, heart rate, pulse rate, and blood pressure, among others.

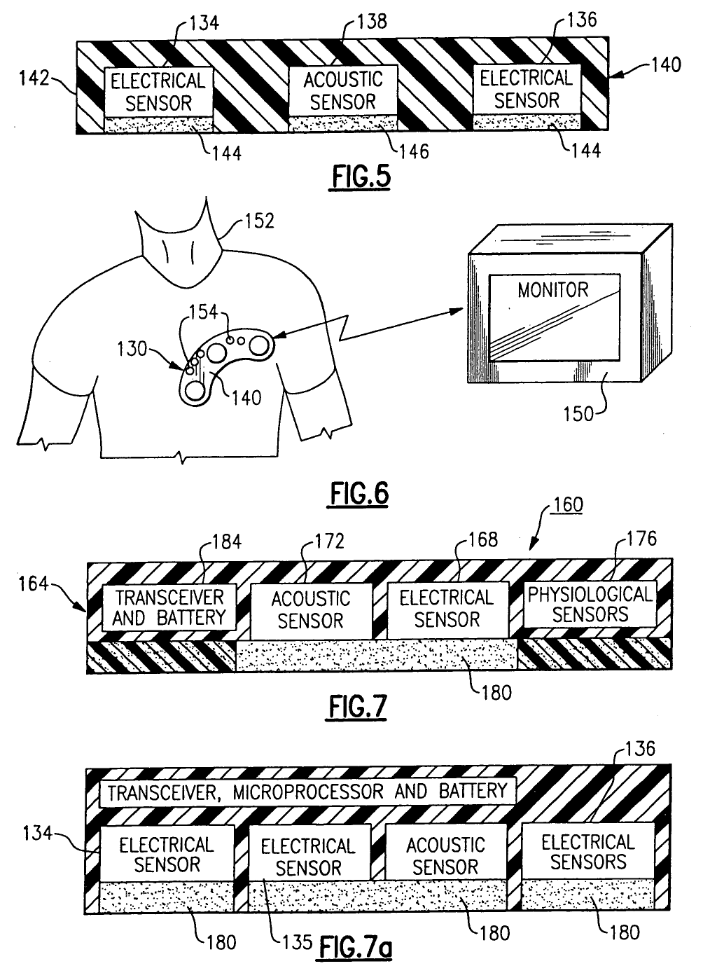

For purposes of background in understanding the problems solved according to the present invention, reference is first made to FIG. 2, in which there is depicted a prior art sensor assembly 45, partially shown, the assembly including an electrical sensor, in this case, an ECG electrode 47 that is embedded within a protective covering 48. The ECG electrode 47 is in the form of an annular ring, that is disposed along the periphery of the bottom of the protective covering 48, also partially shown. The bottom side 52 of the sensor assembly 45 includes an adhesive layer that is peeled for exposure, the ring-like ECG electrode 47 thereby being placed into contact with the skin 51 of a patient. A conductive gel material 55, such as Schiller electrode gel P/N 2.158000 or equivalent, is required for effective electrical contact between the skin of the patient and the sensor.

Still referring to FIG. 2, an acoustic sensor, in this instance, a conventional microphone 60, is separately implanted within the interior of the protective covering 48 of the assembly 45 at the top or apex of a bell-shaped collection volume 64. The collection volume is used to focus respiration (e.g., lung) sounds as well as those relating to the heart. The microphone includes an acoustic transducer, such as an electret sensor, that is disposed at the top of the bell-shaped collection volume. An intermediate air buffer layer is therefore established between the acoustic transducer of the microphone 60 and the skin 51 of the patient within the established collection volume 64.

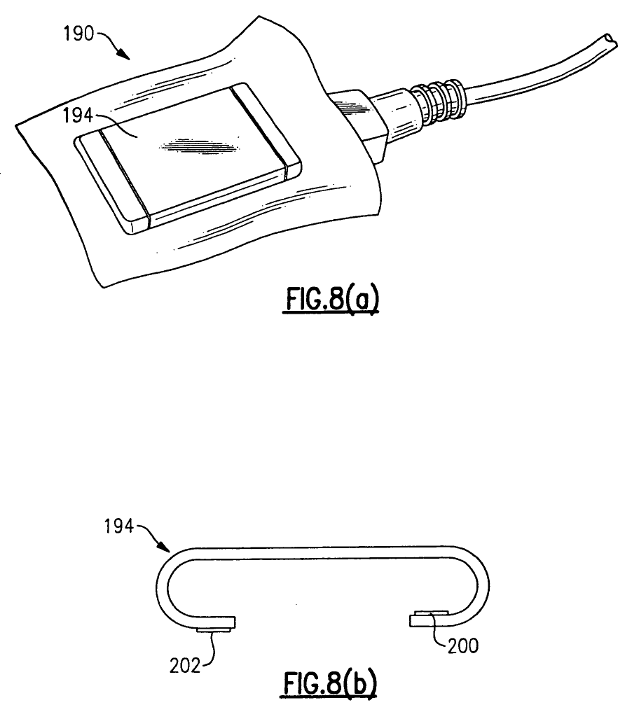

With the preceding background being provided and referring now to FIG. 3, there is shown a combined sensor assembly 80 that is made in accordance with a first embodiment of the present invention. The combined sensor assembly 80 includes a highly flexible enclosure or covering 84 that is made from, a flexible elastomeric material, (such as, for example, medical grade closed cell foam) the covering having a defined upper or top portion 88, as well as a corresponding bottom portion 92. The bottom portion 92 of the herein described assembly 80 includes a foam rubber periphery 96 that is covered by a lower peelable strip (not shown) exposing an adhesive face 100. An interior cavity 104 of the bottom portion 92 of the combined sensor assembly 80 is filled with a gel material 110, such as ECG gel, described in greater detail below.

The top portion 88 of the enclosure 84 of the herein described combined sensor assembly 80 retains a number of retained components. These components include a wireless radio transceiver 114 as well as a portable power supply (such as at least one integrated miniature battery, although the battery can be separately provided), an acoustic sensor 118 (in this instance, an acoustic microphone), and at least one electrical sensor 122 (in this instance, an ECG electrode).

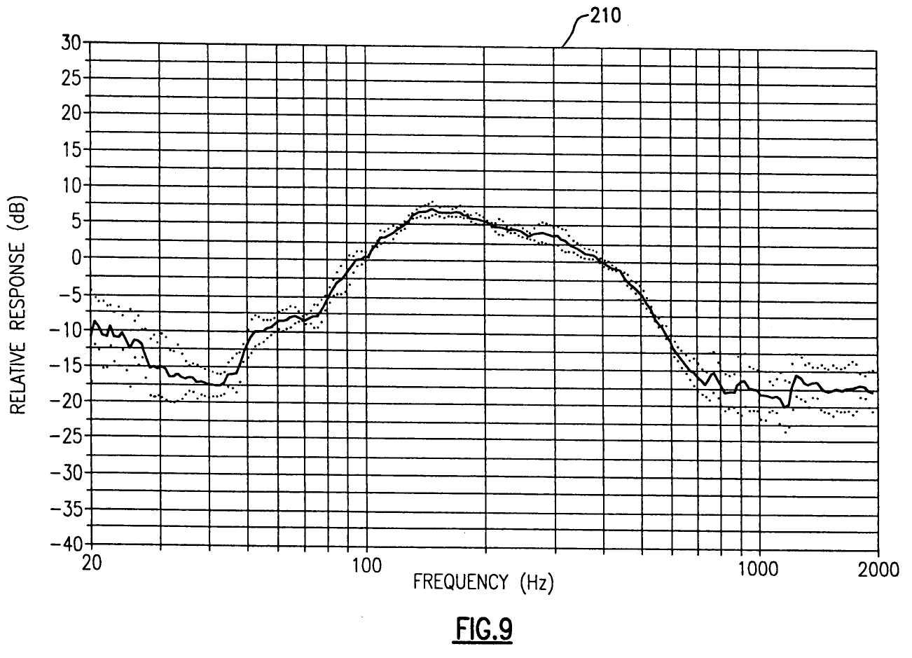

Additional electronic circuitry may be added to the above noted structure 114 as known to those skilled in the art. This circuitry would amplify the signals detected by sensors 122 and 118, digitize them through appropriate A/D converters, manipulate them into usable data information (such as, but not limited to, heart rate and breath rate) via low power microprocessors, and connect the resulting signal and data to the radio transceiver 114. Such microprocessors may also control radio communication links as well. Alternatively, the microprocessors may communicate to an external bedside monitor or system, with wires through connectors 154 (FIG. 4).

For purposes of this embodiment and for reasons of clarity, only a single electrical sensor/electrode is illustrated. As shown in FIG. 3, the acoustic sensor 118 and the electrical sensor 122 are each disposed within a center portion 126 of the top portion 88 of the highly flexible covering 84 and are disposed immediately in relation to the interior cavity 104 containing the gel material 110. According to this embodiment, the acoustic microphone is manufactured by Andromed, Inc., and is defined preferably by a flat or substantially planar piezoelectric transducer, such as described in U.S. Pat. No. 6,661,161B1, the entire contents of which are herein incorporated by reference in their entirety.

In operation, the peelable strip (not shown) of the bottom portion 92 of the combined sensor assembly 80 is removed and the rubber periphery 96 of the combined sensor assembly 80 is attached via the adhesive face 100 directly to the skin of the patient. In this instance, the combined sensor assembly 80 is mounted onto the chest of the patient. An adhesive material may be imbedded in the gel material to improve contact and coupling between the skin and electrical sensors 122 and acoustic sensor 118. The gel material 110 is selected not only to provide an effective electrical contact between the skin of the patient and the electrical sensor 122, but also to provide an effective acoustic impedance match between the flat piezoelectric transducer of the acoustic microphone (acoustic sensor 118) and the skin of the patient. Moreover and based on the design of the sensor assembly 80, there is substantially no air buffer layer provided between the gel material 110 and the flat piezoelectric transducer of the acoustic sensor 118. Other sensor designs can be contemplated wherein the gel material can be either directly added onto the skin of the patient or alternatively, the gel material can also be included within the covering itself at the sensor interface to provide the necessary interconnection, both electrically and acoustically.

The electrical sensor (ECG electrode) 122 operates to detect electrical signals from the heart of the patient and to transmit these signals to a contained miniature microprocessor having sufficient memory for storage. In addition, the miniature microprocessor can further include logic for initially processing the signals. An A/D converter is used to convert the analog sensor signals into a digital format for transmission by the wireless transceiver 114, the transceiver including an antenna. Alternatively, the signals can be transmitted by means of a wired connection to a monitor or other device, wither for processing or for display thereof.

The acoustic portion of the herein described sensor assembly 80 involves vibration of the transducer's piezoelectric material in response to sounds that are produced by the heart, lungs, or vocal cords. This vibration generates voltage across the piezoelectric material and, thereby, an electrical signal representing the sound(s) is also generated. The gel material 110 acts as an acoustic impedance matching (acoustically conductive) medium, thereby providing good transmission of the patient's heart and lung sounds to the piezoelectric material. The acoustic signals are then also either transmitted to the contained microprocessor for storage and/or processing or for transmission using the wireless transceiver 114 to a separate site after converting the signals from an analog to a digital form. According to a preferred embodiment, the herein described sensor assembly 80 can include a multiplexor for incorporating the individual signals, using frequency hopping or other means, into a transmission data packet for transmission using an industry standards-based protocol such as WiFi, 802.11(a,b,g), Ultra Wide Band, Bluetooth, 802.15.1, Zigbee, 802.15.4, or other forms of wireless link. Alternatively, the signals can be transmitted by a wired connection to a separate monitoring device, such as an ECG or other form of monitor, a display, a remote monitoring station or other site.

A myriad of other embodiments are possible within the inventive scope of the invention that has already been already described herein. The following pertains to examples of these embodiments.

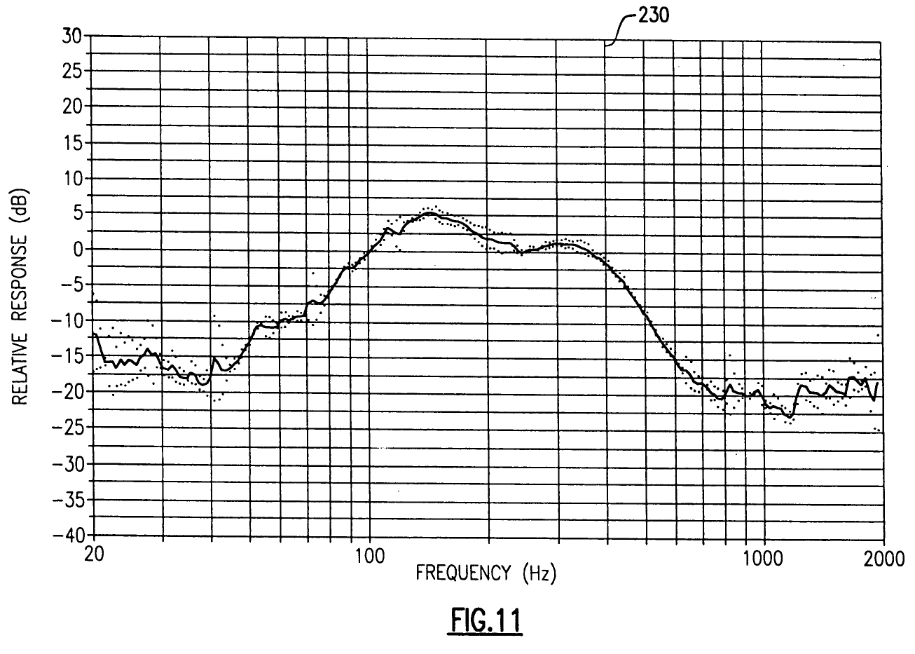

Referring to FIGS. 4-6, a combined sensor assembly 130 made in accordance with a second embodiment of the present invention includes a pair of physiological parameter sensors, in this case, electrical sensors 134, 136, in this case ECG electrodes, each of which are disposed in an elongate substrate 140 and on opposite ends thereof. Preferably, the elongate substrate 140 is made from a highly flexible electrically non-conductive material and is shaped and sized to retain a predetermined number of physiological sensors disposed therein, including those capable of detecting electrical signals relating to the heart for determining ECG. In this instance, the substrate 140 is substantially thin-walled and is crescent shaped to properly fit the ECG electrodes relative to predetermined anatomical positions about the heart of the patient. In addition, at least one acoustic sensor 138, such as an acoustic microphone, is also disposed in the flexible elongate substrate 140. In this embodiment, the acoustic sensor 138 is disposed preferably between the two electrical sensors 134, 136, the microphone preferably having a flat piezoelectric transducer, such as that described by previously incorporated U.S. Pat. No. 6,661,161B1. Additionally, the elongate substrate 140 includes multiple ports 154 adapted to receive leads (not shown) interconnecting the substrate to a monitor 150, as shown in FIG. 6, the assembly 130 being attached to the chest of patient 152.

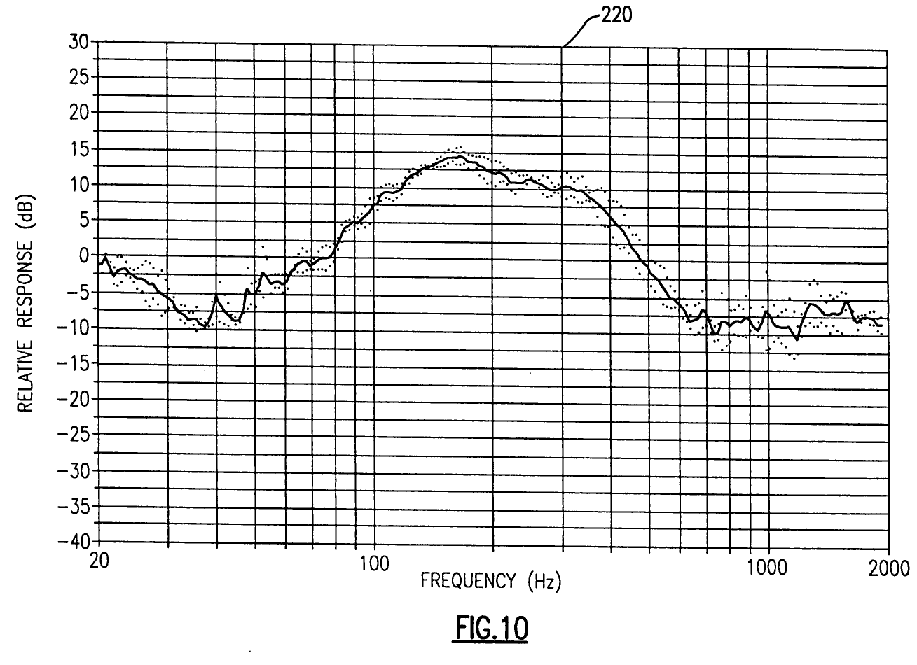

Referring to FIG. 5, it can be shown that each of the electrical sensors 134, 136, can utilize a first conductive gel material 144 in the interface between the sensor and the skin of the patient (not shown) that is electrically conductive, while the acoustic sensor 138 can utilize a different second conductive gel material 146 that is acoustically conductive, the second conductive gel also being provided at the transducer/skin interface. Alternatively, each of the retained physiologic sensors 134, 136, and 138 can utilize or share the same conductive gel material with physical separation of the gel between the sensors. In such an embodiment, the gel would have conductive material characteristics that can be utilized by each of the sensors.

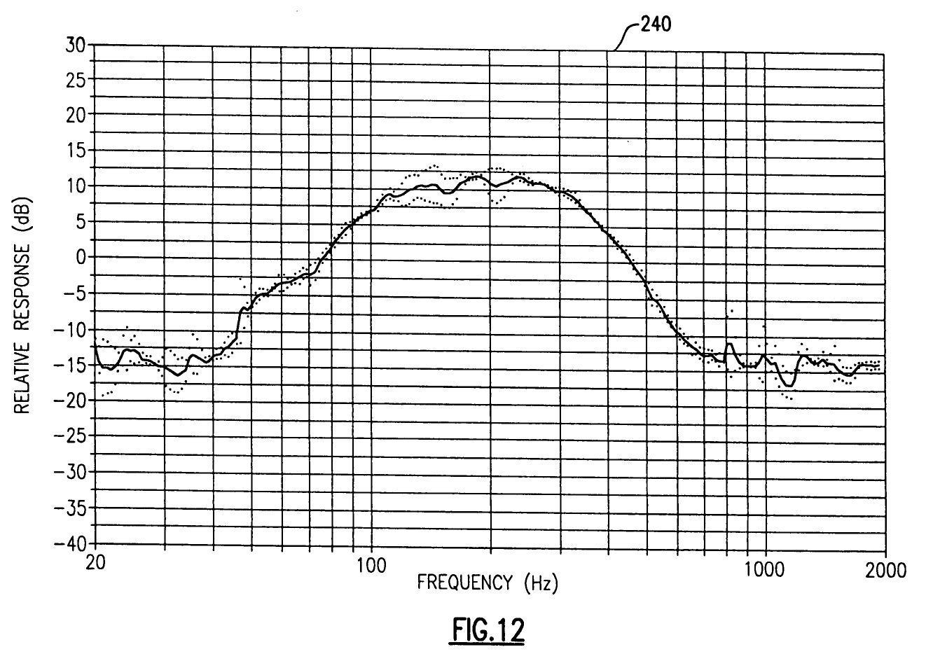

Referring to FIG. 7, there is illustrated a combined sensor assembly 160 for use according to a third embodiment of the present invention. The combined sensor assembly 160 according to this embodiment includes a flexible protective covering 164 made from a flexible elastomeric material, such as, for example, medical grade closed cell foam, that encloses a number of components. These components include at least one electrical sensor 168, in this case at least one ECG electrode, an acoustic sensor 172 (such as a microphone), as well as at least one other physiological parameter measuring sensor 176 capable of measuring body temperature, blood pressure, and the like which does not necessarily rely upon an electrical or acoustical signal from the patient. Alternatively and in lieu of a microphone, other forms of acoustic sensors (such as, for example, electret microphones) can also be used, provided the conductive gel material is located at the interface between the sensor transducer and the skin of the patient in order to substantially eliminate the air buffer. As in the preceding, the acoustic sensor 172 preferably includes a flat piezoelectric transducer wherein each of the electrical sensor 168 and the acoustic sensor 172 are disposed in a center portion of the combined sensor assembly 160 in relation to a bottom side that includes a conductive gel material 180. This conductive gel material 180 is selected to electrically couple to the skin of a patient (not shown), as well as to provide an acoustic impedance match between the flat piezoelectric transducer of the acoustic sensor 172 and the skin of the patient. A wireless transceiver 184, that includes a transmitter and a receiver, is also disposed within the covering 164, as well as a miniature integrated battery used for powering each of the contained components of the combined sensor assembly 160. Alternatively and referring to FIG. 7(a), three(s) electrical sensors are positioned such that the outer two sensors 134, 136 provide a differential biopotential for the sensing of an ECG signal, while the center electrical sensor 135 provides a reference or driven lead to improve signal-to-noise ratio and common node rejection as is known to those skilled in the art. The conductive gel material 180 may be shared by acoustic sensor 138 in a lateral configuration.

In operation, the bottom side of the combined sensor assembly 160 is attached to the skin of the patient and the conductive gel material 180 on the bottom facing side thereof provides both electrical connectivity between the electrical sensor 168 and the skin as well as an acoustic impedance match between the skin and the transducer of the acoustic sensor 172. As in the preceding, there is no intermediate air buffer layer between the transducer of the acoustic sensor 172 and the gel layer 180.

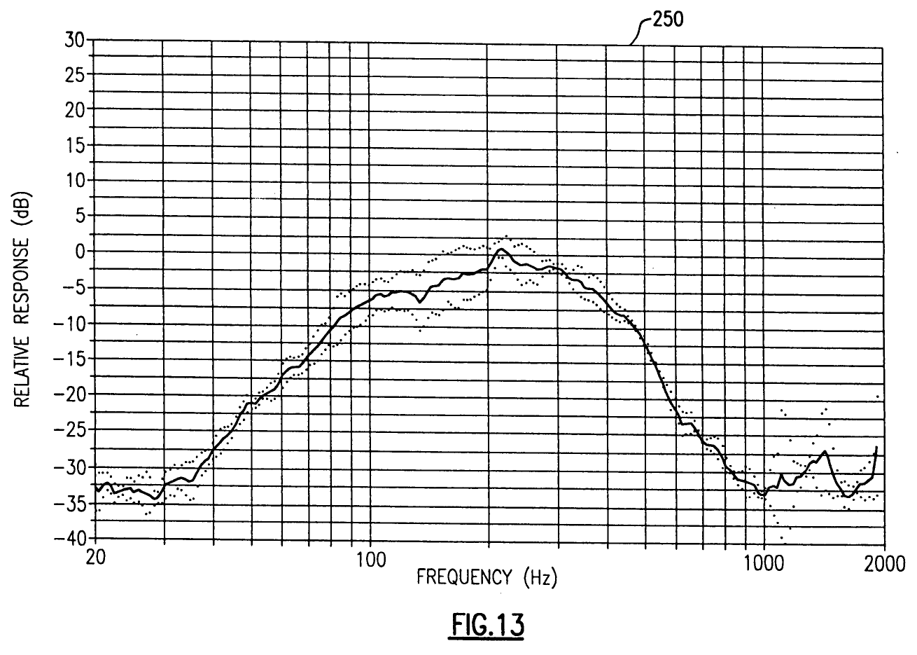

Referring to FIGS. 8(a) and 8(b), there is shown an exemplary acoustic sensor 190 used for purposes of testing. The tests were conducted using a custom designed stethoscope test machine. This test machine comprises a vertically oriented actuator whose output oscillates sinusoidally; an elastomeric pad on the actuator output that simulates the acoustic characteristics of the chest tissue; and a computer that controls the actuator, reads the output signal, and displays and stores the measured signal from the sensor. In operation, the tested sensor 190 is loaded against the elastomeric pad and the frequency of the actuator is swept from 20 Hz to 2000 Hz. The sensor 190 used for purposes of this test is manufactured by Andromed in accordance with previously incorporated U.S. Pat. No. 6,661,161B1 and includes a thin piezoelectric film or membrane 194 provided on the exterior (patient facing side) of the sensor, the interior including a printed circuit board (PCB) (not shown). Electrical contact is established between the exterior of the acoustic sensor 190 and the printed circuit board (not shown) in the interior of the acoustic sensor by means of electrical coatings 200, 202 provided on opposite sides of the piezoelectric film or membrane 194, as shown in FIG. 8(b). The detection of voltage and/or current is made using these opposed electrical coatings, the voltage being produced by the imposition of a mechanical motion (e.g., an applied respiratory sound) on the sensor. That is to say, acoustically produced motions in the sensor will produce a corresponding electric signal that is detected by a circuit of the sensor contained in the PCB.

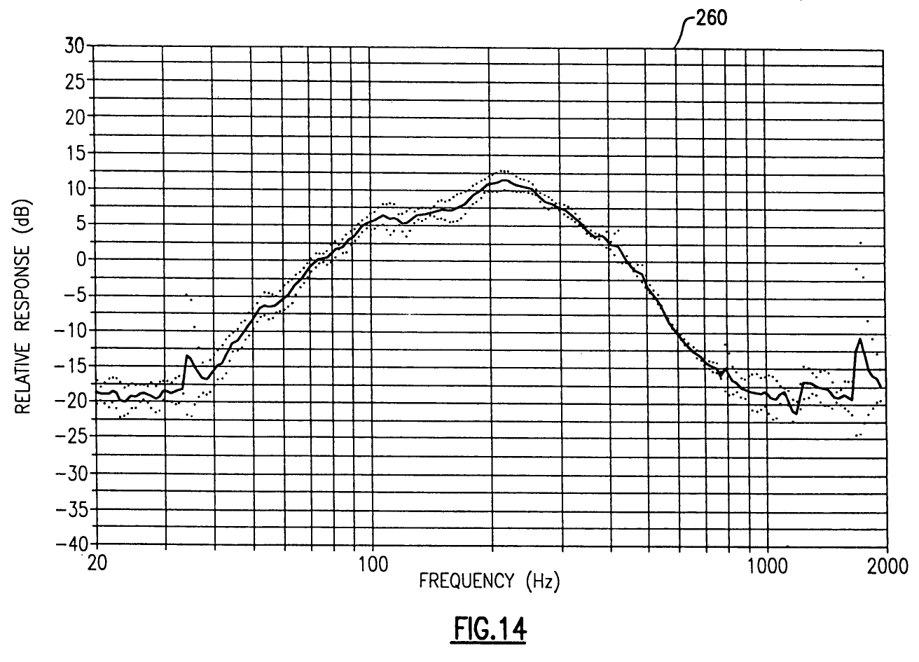

Referring to FIGS. 9-14, there are represented a series of individual plots 210, 220, 230, 240, 250, 260 using the acoustic sensor of FIGS. 8(a) and 8(b). The plots show the measured signal (dB) from the sensor versus actuator frequency, measured in Hertz, for various applied loads. Accordingly, six (6) tests were conducted using a total of three different loads (0.5 kg, 0.3 kg, 0.1 kg) between the acoustic sensor and the skin surface, which was simulated by the elastomeric pad of the above-described stethoscope tester. At each load, the tests compared the use of a conductive gel material at the sensor/tester interface with no gel (e.g., air at the interface). The results of the tests according to FIGS. 9 (no gel) and 10 (with gel), at which the applied load was 0.5 kg indicated comparatively that an approximate 5 dB signal increase over much of the curve occurs with conductive gel material added. This increase represents a factor of approximately 3 increase in signal energy.

FIGS. 11 (no gel) and 12 (with gel) provide similar representations at 0.3 kg with the comparative results, indicating that the signal difference between the two plots averages approximately 7 dB over much of the curve. This increase represents a factor of nearly 5 increase in signal energy for this load.

Finally, FIGS. 13 (no gel) and 14 (with gel) represent air/gel curves, respectively, taken at 0.1 kg. The results at this load indicate a signal difference of nearly 12 dB associated with adding gel to the sensor/tester interface or a factor increase of about 16 in signal energy. As a result, it appears the results of using conductive gel are more profound with decreased or minimal loads though an increase was demonstrated at each load.

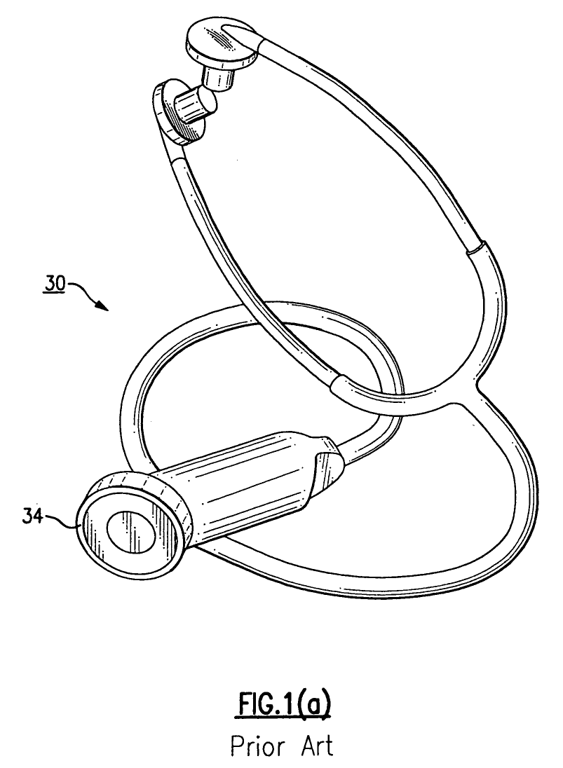

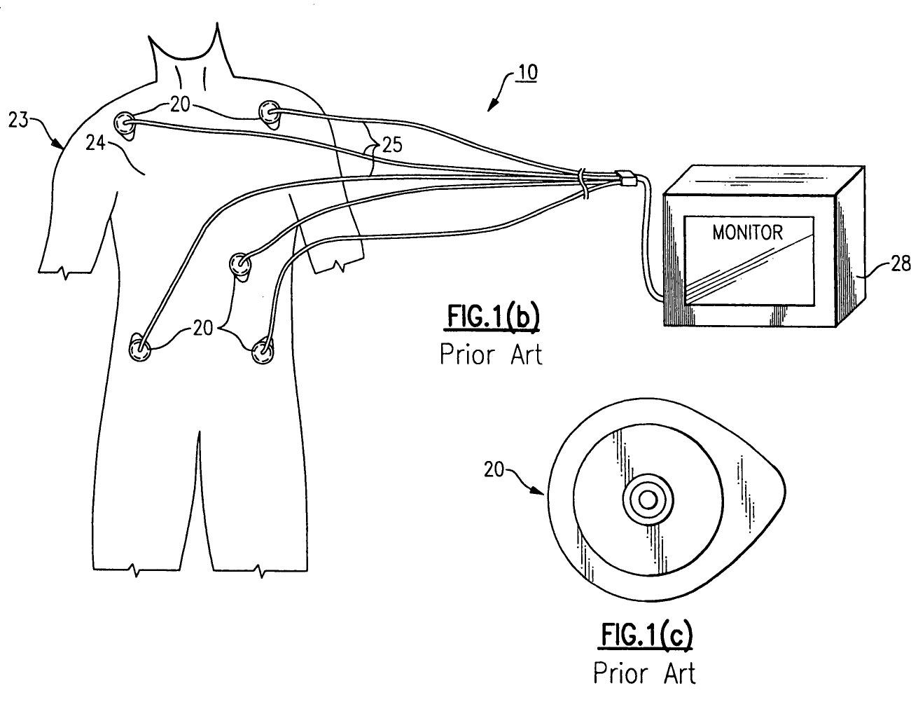

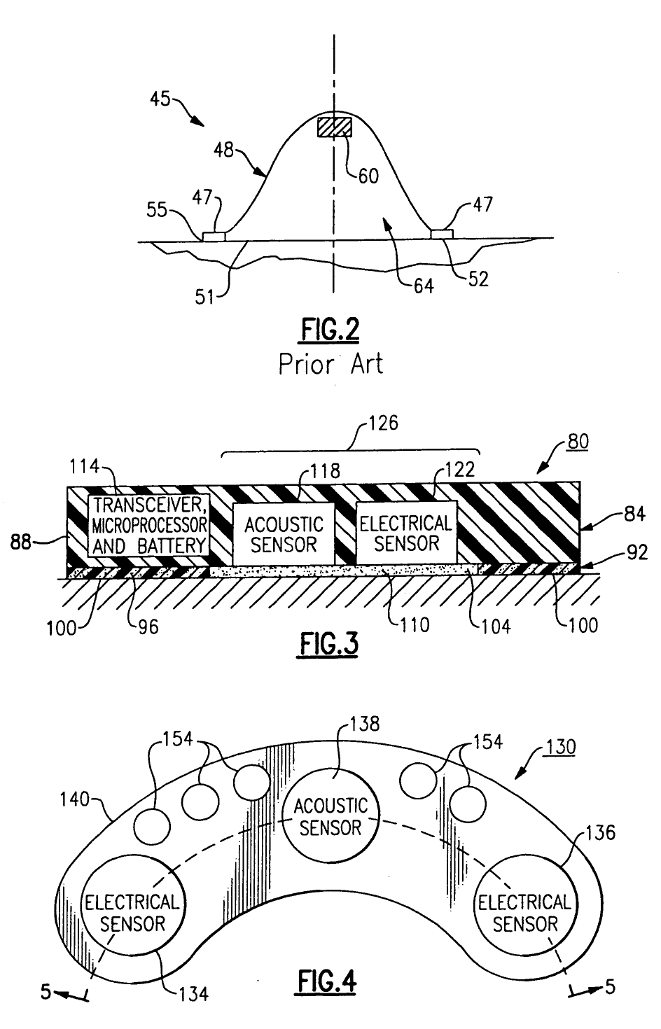

### PARTS LIST FOR FIGS. 1-14

| | |
|---|---|
| 10 | sensor assembly |
| 20 | electrodes |
| 23 | patient |
| 24 | chest |
| 25 | cables |
| 28 | monitor |
| 30 | stethoscope |
| 34 | transducer, acoustic |
| 45 | sensor assembly |
| 47 | ECG electrode |
| 48 | protective covering |
| 51 | skin |
| 52 | bottom side |
| 55 | conductive gel material |
| 60 | microphone |
| 64 | collection volume |
| 80 | combined sensor assembly |
| 84 | covering |
| 88 | top portion |
| 92 | bottom portion |
| 96 | foam rubber periphery |
| 100 | adhesive face |
| 104 | interior cavity |
| 110 | gel material |
| 114 | wireless transceiver |
| 118 | acoustic sensor |
| 122 | electrical sensor |
| 126 | center portion |
| 130 | assembly, combined sensor |
| 134 | electrical sensor |
| 135 | center electrical sensor |
| 136 | electrical sensor |
| 138 | acoustic sensor |
| 140 | elongate substrate |
| 144 | first conductive gel |
| 146 | second conductive gel |
| 150 | monitor |
| 152 | patient |
| 154 | ports |
| 160 | combined sensor assembly |
| 164 | protective covering |
| 168 | electrical sensor |
| 172 | acoustic sensor |
| 176 | physiological parameter sensor |
| 180 | conductive gel material |
| 184 | wireless transceiver |
| 190 | acoustic sensor |
| 194 | piezoelectric film or membrane |
| 200 | electrical coating |
| 202 | electrical coating |
| 210 | plot (.5 kg, no gel) |
| 220 | plot (.5 kg, with gel) |
| 230 | plot (.3 kg, no gel) |
| 240 | plot (.3 kg, with gel) |
| 250 | plot (.1 kg, no gel) |
| 260 | plot (.1 kg, with gel) |

It will be readily apparent from the foregoing discussion, that numerous modifications and variations are possible to one of adequate skill in the field that will embody the inventive concepts capturing the scope of the invention, as now posited by the following claims.

We claim:

1. A combined physiological sensor assembly comprising:
at least one electrical sensor, said at least one electrical sensor being capable of measuring electrical signals representative of a physiological parameter of a patient and coupled thereto by means of an electrically conductive gel material; and
at least one acoustic sensor, each said at least one acoustic sensor being coupled to a patient by means of an acoustically conductive gel material.

2. A combined sensor assembly as recited in claim 1, wherein said at least one sensor measures ECG electrical signals from the heart.

3. A combined sensor assembly as recited in claim 1, wherein the acoustically conductive gel material and the electrically conductive gel material are the same gel material.

4. A combined sensor assembly as recited in claim 1, wherein said at least one acoustic sensor comprises a microphone.

5. A combined sensor assembly as recited in claim 4, wherein said microphone includes a substantially flat piezoelectric transducer.

6. A combined sensor assembly as recited in claim 5, wherein said transducer is disposed in immediate proximity to said acoustically conductive gel material.

7. A combined sensor assembly as recited in claim 1, wherein said assembly includes a covering, said at least one electrical sensor and said at least one acoustic sensor being disposed within said covering.

8. A combined sensor assembly as recited in claim 7, wherein said covering is made from a highly flexible material.

9. A combined sensor assembly as recited in claim 1, wherein at least a portion of said assembly is disposable.

10. A combined sensor assembly as recited in claim 1, including at least one of a wired and a wireless transceiver for transmitting signals between at least one of said at least one electrical sensor and said at least one acoustic sensor and at least one separate station.

11. A combined sensor assembly as recited in claim 4, including at least one of a wired and a wireless transceiver for transmitting signals between at least one of said at least one electrical sensor and said microphone and at least one separate station.

12. A combined sensor assembly as recited in claim 1, wherein said acoustically conductive gel material is different than the electrically conductive gel material.

13. A combined sensor assembly as recited in claim 1, including at least two electrical sensors, said at least two sensors being spaced from one another.

14. A combined sensor assembly as recited in claim 1, including at least one other physiological parameter measuring sensor.

15. A combined sensor assembly as recited in claim 14, wherein said at least one other physiological sensor does not utilize electrical or acoustic signal input.

16. A combined sensor assembly as recited in claim 1, wherein said at least one acoustic sensor includes a transducer that is directly coupled to said acoustically conductive gel material without air therebetween.

17. A combined sensor assembly as recited in claim 6, wherein said transducer, said acoustically conductive gel material and the skin of the patient defines an interface region, said interface region being essentially devoid of air.

18. A method for monitoring a patient, said method comprising:
disposing at least one electrical sensor capable of measuring electrical signals representative of a physiological parameter of a patient coupling said at least one electrical sensor to said patient using an electrically conductive gel material;
disposing at least one acoustic sensor in relation to said at least one electrical sensor; and
coupling said at least one acoustic sensor to said patient using an acoustically conductive gel material.

19. A method as recited in claim 18, wherein said acoustically conductive gel material and said electrically conductive gel material is the same gel material.

20. A method as recited in claim 18, wherein said acoustically conductive gel material and said electrically conductive gel material is a different gel material.

21. A method as recited in claim 18, wherein said at least one acoustic sensor includes a planar transducer, said transducer being placed in relation to said acoustically conductive gel material without an air buffer therebetween.

22. A method as recited in claim 18, wherein said at least one acoustic sensor is a microphone.

23. A method as recited in claim 18, including the step of transmitting signals via wires from said at least one acoustic sensor and said at least one electrical sensor to a separate location.

24. A method as recited in claim 18, including the step of wirelessly transmitting signals from said at least one acoustic sensor and said at least one electrical sensor to a separate location.

25. A method as recited in claim 18, wherein said at least one electrical sensor is an ECG electrode.

26. A method as recited in claim 18, including the step of disposing at least one additional physiological sensor in relation to said patient.
